(19) United States
(12) Patent Application Publication
(10) Pub. No.: US 2024/0207535 A1
(43) Pub. Date: Jun. 27, 2024

(54) DRUG INJECTOR ADAPTER

(71) Applicant: JSKBIOMED INC., Daejeon (KR)

(72) Inventors: Sukbae SEO, Seoul (KR); Jinwoo JEON, Sejong-si (KR)

(73) Assignee: JSKBIOMED INC., Daejeon (KR)

(21) Appl. No.: 18/288,723

(22) PCT Filed: Feb. 3, 2022
(86) PCT No.: PCT/KR2022/001716

§ 371 Date: Oct. 27, 2023

(30) Foreign Application Priority Data
Apr. 30, 2021 (KR) 10-2021-0056728

(51) Int. Cl. A61M 5/34

(52) U.S. Cl. CPC A61M 5/345

(57) ABSTRACT

Provided is a drug injector adapter configured to be attachable to and detachable from a needleless drug injector, the drug injector adapter which comprises a fixing portion that is inserted and fixed in a position where a nozzle of the drug injector was removed, a coupling portion having a structure for coupling a needle to an opposite side of the fixing portion and a delivery flow passage configured to deliver drugs delivered from the drug injector to the needle in a state in which the drug injector, the needle, and the drug injector adapter are coupled to each other.

### TECHNICAL FIELD

The present invention relates to a drug injector adapter, and more particularly, to a drug injector adapter that is configured to be attachable to and detachable from a needleless drug injector and has a structure that allows a needle to be coupled to the needleless drug injector.

### BACKGROUND ART

A drug delivery system is used to deliver drugs to the human body for various purposes, such as treatment of human diseases, wounds, or beauty, and an injection method using a needle is most often used due to its safety and efficiency.

However, although a drug delivery system using a needle enables accurate and efficient drug administration, it may cause patients to have injection phobia due to pain during injection. In addition, the drug delivery system using a needle also have problems such as the risk of infection due to reuse of needles and the generation of large amounts of medical waste.

Accordingly, development of structures for delivering drugs to the human body without a needle continues. However, because the structures for delivering drugs to the human body without a needle also have pros and cons, there is still a need for a structure capable of mixing a needle as needed.

### DESCRIPTION OF EMBODIMENTS

### Technical Problem

The present invention provides a drug injector adapter that is configured to be attachable to and detachable from a needleless drug injector and has a structure that allows a needle to be coupled to the needleless drug injector.

### Solution to Problem

According to an aspect of an embodiment, a drug injector adapter configured to be attachable to and detachable from a needleless drug injector, the drug injector adapter may comprise a fixing portion that is inserted and fixed in a position where a nozzle of the drug injector was removed, a coupling portion having a structure for coupling a needle to an opposite side of the fixing portion and a delivery flow passage configured to deliver drugs delivered from the drug injector to the needle in a state in which the drug injector, the needle, and the drug injector adapter are coupled to each other.

According to an aspect of an embodiment, one side of the delivery flow passage may be connected to a flow passage of the drug injector according to the coupling with the drug injector.

According to an aspect of an embodiment, the other side of the delivery flow passage may be connected to a flow passage of the needle according to the coupling with the needle.

According to an aspect of an embodiment, the drug injector adapter may further comprise a first sealing portion configured to allow the fixing portion to come into close contact with the drug injector.

According to an aspect of an embodiment, the first sealing portion may be in close contact with the delivery flow passage of the drug injector adapter and the flow passage of the drug injector to connect them in a tightly sealed state when the drug injector adapter is coupled to the drug injector.

According to an aspect of an embodiment, the first sealing portion may be formed of an elastic material.

According to an aspect of an embodiment, the drug injector adapter may further comprise a support portion configured to be in close contact with an outer surface of the drug injector in a state where the fixing portion is inserted into the position where the nozzle of the drug injector was removed.

According to an aspect of an embodiment, the drug injector adapter may further comprise an adapter housing configured to surround the coupling portion and constitute a coupling groove into which the needle is inserted and coupled.

According to an aspect of an embodiment, the adapter housing may comprise a second sealing portion that is in close contact with the delivery flow passage of the drug injector adapter and the flow passage of the needle to connect them in a tightly sealed state when the drug injector adapter is coupled to the needle.

According to an aspect of an embodiment, the coupling portion may be formed in a protruding structure corresponding to an insertion groove of the needle and is configured to be coupled to the needle.

### Advantageous Effects of Disclosure

A drug injector adapter according to an embodiment of the present invention is configured to be attachable to and detachable from a needleless drug injector and has a structure that allows a needle to be coupled to the needleless drug injector, so that the type of drug injector may be changed as needed.

In addition, a drug injector adapter according to an embodiment of the present invention may be configured so that various types of needles may be coupled to a needleless drug injector.

### BRIEF DESCRIPTION OF DRAWINGS

A brief description of each drawing is provided to more fully understand drawings recited in the detailed description of the present invention.

FIG. 1 is a perspective view of an embodiment of a needleless drug injector to which a drug injector adapter can be coupled, according to an embodiment of the present invention.

FIG. 2 is an exploded view of the drug injector shown in FIG. 1.

FIG. 3 is a cross-sectional view of the drug injector shown in FIG. 1.

FIGS. 4 and 5 are views illustrating an exemplary form in which a drug injector adapter and a needle according to an embodiment of the present invention are attached to and detached from the drug injector shown in FIG. 1.

### MODE OF DISCLOSURE

Since the disclosure may have diverse modified embodiments, preferred embodiments are illustrated in the drawings and are described in the detailed description. However, this is not intended to limit the disclosure to particular modes of practice, and it is to be appreciated that all changes, equivalents, and substitutes that do not depart from the spirit and technical scope of the disclosure are encompassed in the disclosure.

In the description of the disclosure, certain detailed explanations of the related art are omitted when it is deemed that they may unnecessarily obscure the essence of the disclosure. In addition, numeral figures (e.g., first, second, and the like) used during describing the specification are just identification symbols for distinguishing one element from another element.

Further, in the specification, if it is described that one component is “connected” or “accesses” the other component, it is understood that the one component may be directly connected to or may directly access the other component but unless explicitly described to the contrary, another component may be “connected” or “access” between the components.

In addition, terms including “unit,” “er,” “or,” “module,” and the like disclosed in the specification mean a unit that processes at least one function or operation and this may be implemented by hardware or software such as a processor, a micro processor, a micro controller, a central processing unit (CPU), a graphics processing unit (GPU), an accelerated Processing unit (APU), a digital signal processor (DSP), an application specific integrated circuit (ASIC), and a field programmable gate array (FPGA) or a combination of hardware and software. Furthermore, the terms may be implemented in a form coupled to a memory that stores data necessary for processing at least one function or operation.

In addition, it is intended to clarify that the division of the components in the specification is only made for each main function that each component is responsible for. That is, two or more components to be described later below may be combined into one component, or one components may be divided into two or more components according to more subdivided functions. In addition, it goes without saying that each of the components to be described later below may additionally perform some or all of the functions of other components in addition to its own main function, and some of the main functions that each of the components is responsible for may be dedicated and performed by other components.

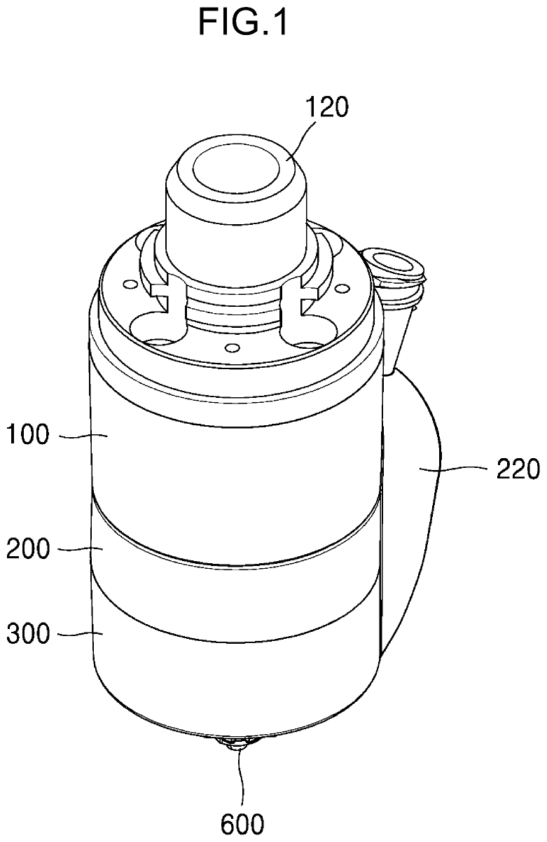

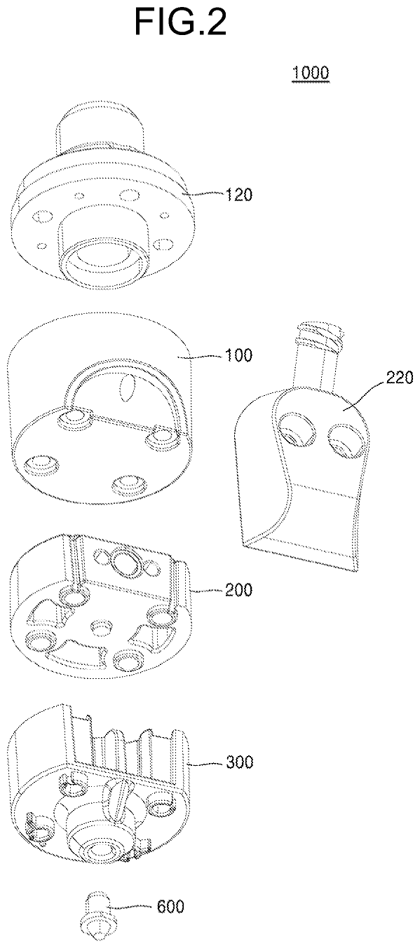

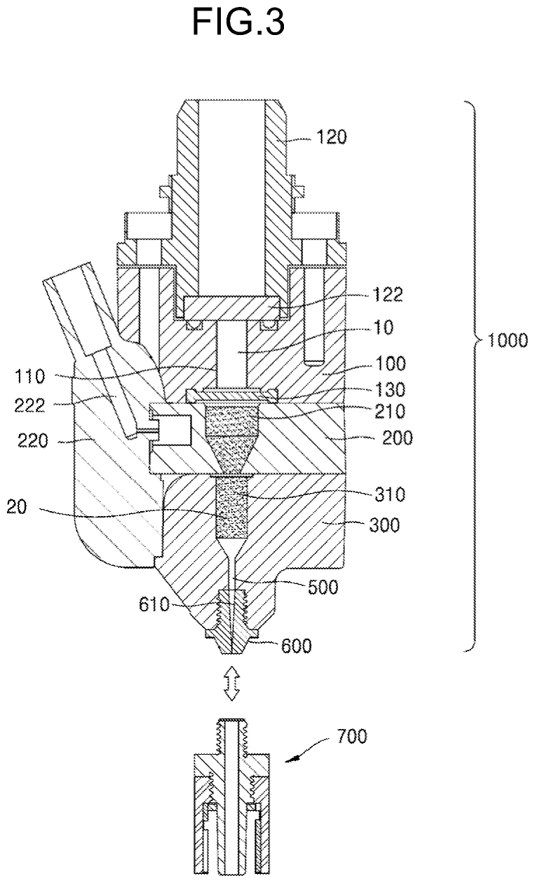

FIG. 1 is a perspective view of an embodiment of a needless drug injector to which a drug injector adapter can be coupled, according to an embodiment of the present invention. FIG. 2 is an exploded view of the drug injector shown in FIG. 1. FIG. 3 is a cross-sectional view of the drug injector shown in FIG. 1.

Referring to FIGS. 1 to 3, a needleless drug injector 1000 to which a drug injector adapter can be coupled, according to an embodiment of the present invention, may include a first housing 100, a pressure generating unit 120, a second housing 200, an injector 220, a chamber 300, and a nozzle 600.

A first storage portion 110 inside the first housing 100 may be filled with pressure generating liquid 10.

The pressure generating unit 120 is located at the top of the first storage portion 110 to maintain a sealed state of the first storage portion 110, and may concentrate and apply energy toward the pressure generating liquid 10.

According to an embodiment, the pressure generating unit 120 may generate a laser beam toward the pressure generating liquid 10. In this case, the pressure generating unit 120 may be implemented as a laser beam generating device. At this time, the first housing 100 may include an optical system 122. The optical system 122 may integrate the laser beam generated by the pressure generating unit 120 and transmit it to the pressure generating liquid 10.

The pressure generating liquid 10 may expand in volume by energy (e.g., laser beam) applied from the pressure generating unit 120. An elastic membrane 130 is stretched according to the volume expansion of the pressure generating liquid 10. According to the extension of the elastic membrane 130, a drug solution 20 stored in a second storage portion 210 and a third storage portion 310, which will be described later, may be injected through a flow passage 500 and an injection flow passage 610 of a nozzle 600.

According to an embodiment, the elastic membrane 130 may be made of various materials with elasticity and may have various forms capable of transmitting pressure resulting from the expansion of the pressure generating liquid 10 to a lower part.

According to an embodiment, the pressure generating liquid 10 may be implemented as a reflector, a liquid mixed with opaque materials, or an opaque liquid. Accordingly, because high energy (e.g., a laser beam) generated from the pressure generating unit 120 penetrates the elastic membrane 130 and reaches the drug solution 20, the drug solution 20 may be prevented from being deteriorated.

According to an embodiment, the pressure generating liquid 10 may be implemented as various liquids such as water, alcohol, or polyethylene glycol. According to an embodiment, the pressure generating liquid 10 may be implemented as a liquid material of various shapes such as sol or gel.

The second housing 200 is coupled to a lower part of the first housing 100, and the second storage portion 210 filled with the drug solution 20 may be formed inside the second housing 200.

The drug solution 20 may refer to a drug to be injected into the human body through the drug injector 1000, a drug injector adapter 700, and a needle 800 according to an embodiment of the present invention.

The second storage portion 210 may be formed in a hollow form with open upper and lower parts. The upper part of the second storage part 210 may be sealed by the elastic membrane 130, and the lower part of the second storage part 210 may be in communication with the third storage part 310 of the chamber 300.

The injector 220 may be coupled to the outside of the second housing 200.

The side of the second storage portion 210 may have a fluid migration path communicating with an injection passage 222 of the injector 220.

The drug solution 20 may be supplied to the second storage portion 210 and the third storage portion 310 through the injection passage 222 of the injector 220.

According to an embodiment, when at least a portion of the drug solution 20 is injected, the injector 220 supplies the drug solution 20 so that the second storage portion 210 may be filled with the drug solution 20 by the amount injected.

The chamber 300 is coupled to a lower part of the second housing 200, and the third storage portion 210 filled with the drug solution 20 may be formed inside the chamber 300.

The flow passage 500 that forms a migration path for the drug solution 20 pushed out from the third storage portion 310 may be provided at a lower part of the third storage portion 310.

The drug solution 20 moved through the flow passage 500 may be injected through the injection flow passage 610 of the nozzle 600.

That is, the needleless drug injector 1000 to which the drug injector adapter 700 can be coupled according to the embodiment of the present invention has a structure capable of injecting drugs into the human body without a needle.

The structure of the drug injector 1000 shown in FIG. 1 is only an exemplary structure, and may have various forms capable of injecting drugs into the human body without a needle.

The drug injector adapter 700 according to an embodiment of the present invention may be fixed while being inserted into a position where the nozzle 600 was removed.

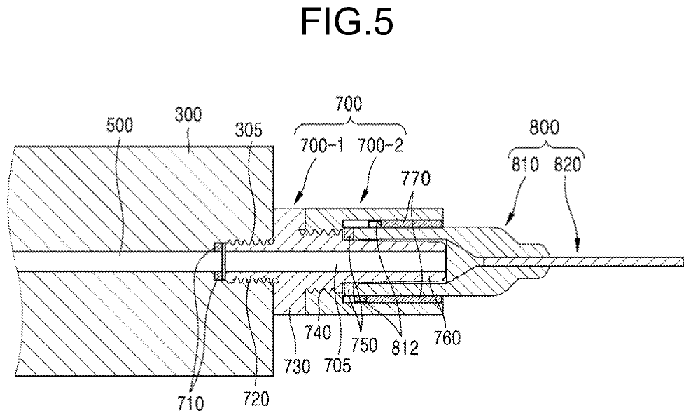

According to an embodiment, a portion (e.g., a fixing portion 720 in FIG. 5) inserted from the drug injector adapter 700 into the drug injector 1000 may be implemented in a size and structure corresponding to a portion inserted from the nozzle 600 into the drug injector 1000.

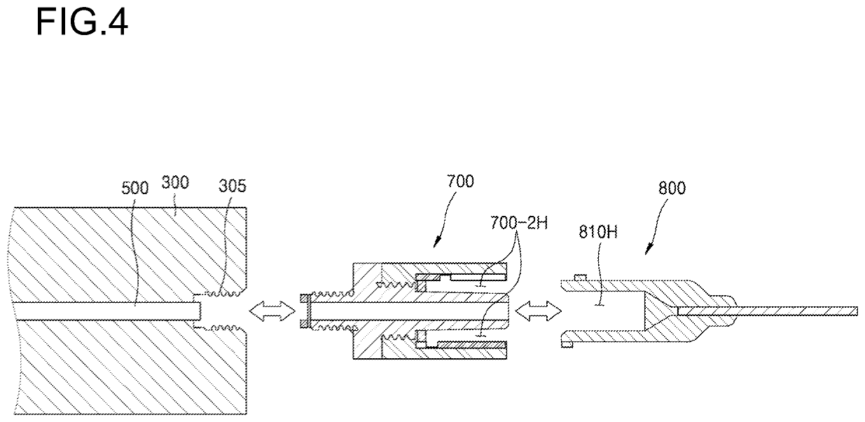

The structure to which the drug injector adapter 700 is coupled will be described later with reference to FIGS. 4 and 5.

FIGS. 4 and 5 are views illustrating an exemplary form in which a drug injector adapter and a needle according to an embodiment of the present invention are attached to and detached from the drug injector shown in FIG. 1.

Referring to FIGS. 1 to 4, when the nozzle 600 is removed from the chamber 300 of the drug injector 1000, an insertion groove 305 into which the nozzle 600 was inserted is exposed.

At this time, one side of the drug injector adapter 700 may be inserted into and fixed to the insertion groove 305, and the other side of the drug injector adapter 700 may be coupled to the needle 800.

Referring to FIG. 5 together, the drug injector adapter 700 may include a body 700-1 and an adapter housing 700-2.

The body 700-1 of the drug injector adapter 700 may include a delivery flow passage 705, a first sealing portion 710, a fixing portion 720, a support portion 730, a first coupling portion 740, a second sealing portion 750, and a second coupling portion 760.

The delivery flow passage 705 may form a path for moving drugs between the flow passage 500 of the drug injector 1000 and a flow passage 820 of the needle 800.

The fixing portion 720 may be inserted into a position (e.g., the insertion groove 305) where the nozzle 600 was removed from the drug injector 1000 to fix the drug injector adapter 700.

According to an embodiment, the fixing portion 720 and the insertion groove 305 may be coupled in various ways, such as a screw coupling method or a fitting coupling method.

The first sealing portion 710 may be formed at the end of the fixing portion 720. The first sealing portion 710 may be configured so that the fixing portion 720 may be in close contact with the insertion groove 305 of the drug injector 1000.

When the drug injector adapter 700 is coupled to the drug injector 1000, the first sealing portion 710 may be in close contact between the fixing portion 720 and the insertion groove 305 to connect the delivery flow passage 705 of the drug injector adapter 700 and the flow passage 500 of the drug injector 100 in a tightly sealed state.

According to an embodiment, the first sealing portion 710 may be formed of an elastic material.

The support portion 730 may be configured to be in close contact with an outer surface of the drug injector 1000 (e.g., an outer surface of the chamber 300) when the fixing portion 720 is inserted into the position (e.g., insertion groove 305) where the nozzle 600 of the drug injector 1000 was removed. The support portion 730 may support the outer surface of the drug injector 1000 (e.g., an outer surface of the chamber 300) to prevent the drug injector adapter 700 from being excessively inserted into the drug injector 1000.

The first coupling portion 740 may have a coupling structure for coupling the adapter housing 700-2.

According to an embodiment, the adapter housing 700-2 may be coupled to the first coupling portion 740 in various ways, such as a screw coupling method or a fitting coupling method.

The second coupling portion 760 may have a structure for coupling the needle 800 to the opposite side of the fixing portion 720, for example, a protruding structure.

According to an embodiment, the needle 800 may be coupled to the second coupling portion 760 in various ways, such as a screw coupling method or a fitting coupling method.

The adapter housing 700-2 surrounds the second coupling portion 760 and may include a coupling groove 700-2H into which the needle 800 can be inserted and coupled.

The coupling groove 700-2H may refer to a space formed between a wall of the adapter housing 700-2 and the second coupling portion 760 of the body 700-1.

A coupling member 770 may be further formed on the wall of the adapter housing 700-2 in the coupling groove 700-2H. The coupling member 770 may firmly fix the needle 800 when it is inserted into the coupling groove 700-2H. According to an embodiment, the coupling member 770 may be formed of an elastic material. According to an embodiment, the coupling member 770 may firmly fix the needle 800 by forming a groove in a portion corresponding to a protrusion 812 of the needle 800.

When the drug injector adapter 700 is coupled to the needle 800, the adapter housing 700-2 may include the second sealing portion 750 that is in close contact with the delivery flow passage 705 of the drug injector adapter 700 and the flow passage 820 of the needle 800 to connect them in a tightly sealed state.

According to an embodiment, the second sealing portion 750 may be formed of an elastic material.

The needle 800 may include a body portion 810 and the flow passage 820.

The body portion 810 of the needle 800 may include an insertion groove 810H into which the second coupling portion 760 of the drug injector adapter 700 can be inserted. The second coupling portion 760 of the drug injector adapter 700 may have a protruding structure corresponding to the insertion groove 810H.

According to an embodiment, the protrusion 812 may be formed on the body portion 810 of the needle 800. At this time, the protrusion 812 may be in close contact with the wall of the adapter housing 700-2 of the drug injector adapter 700 or a groove formed in the coupling member 770, and may be firmly coupled to the drug injector adapter 700.

According to an embodiment, because the drug injector adapter 700 is coupled to the drug injector 1000 and the needle 800, the flow passage 500 of the drug injector 1000, the delivery flow passage 705 of the drug injector adapter 700, and the flow passage 820 of the needle 800 may be connected to each other. Accordingly, a drug solution ejected from the drug injector 1000 may be injected into the human body into which the needle 800 is inserted through the delivery flow passage 705 of the drug injector adapter 700 and the flow passage 820 of the needle 800.

Hereinabove, the present invention has been described with reference to the preferred embodiments. However, it will be appreciated by one of ordinary skill in the art that various modifications and changes of the present invention can be made without departing from the scope of the inventive concept which are defined in the appended claims and their equivalents.

1. A drug injector adapter configured to be attachable to and detachable from a needleless drug injector, the drug injector adapter comprising:
a fixing portion that is inserted and fixed in a position where a nozzle of the drug injector was removed;
a coupling portion having a structure for coupling a needle to an opposite side of the fixing portion; and
a delivery flow passage configured to deliver drugs delivered from the drug injector to the needle in a state in which the drug injector, the needle, and the drug injector adapter are coupled to each other.

2. The drug injector adapter of claim 1, wherein one side of the delivery flow passage is connected to a flow passage of the drug injector according to the coupling with the drug injector.

3. The drug injector adapter of claim 2, wherein the other side of the delivery flow passage is connected to a flow passage of the needle according to the coupling with the needle.

4. The drug injector adapter of claim 3, further comprising:
a first sealing portion configured to allow the fixing portion to come into close contact with the drug injector.

5. The drug injector adapter of claim 4, wherein the first sealing portion is in close contact with the delivery flow passage of the drug injector adapter and the flow passage of the drug injector to connect them in a tightly sealed state when the drug injector adapter is coupled to the drug injector.

6. The drug injector adapter of claim 5, wherein the first sealing portion is formed of an elastic material.

7. The drug injector adapter of claim 6, further comprising:
a support portion configured to be in close contact with an outer surface of the drug injector in a state where the fixing portion is inserted into the position where the nozzle of the drug injector was removed.

8. The drug injector adapter of claim 7, further comprising:
an adapter housing configured to surround the coupling portion and constitute a coupling groove into which the needle is inserted and coupled.

9. The drug injector adapter of claim 8, wherein the adapter housing comprises:
a second sealing portion that is in close contact with the delivery flow passage of the drug injector adapter and the flow passage of the needle to connect them in a tightly sealed state when the drug injector adapter is coupled to the needle.

10. The drug injector adapter of claim 1, wherein the coupling portion is formed in a protruding structure corresponding to an insertion groove of the needle and is configured to be coupled to the needle.
